(19) United States
(12) Patent Application Publication
(10) Pub. No.: US 2011/0077206 A1
(43) Pub. Date: Mar. 31, 2011

(54) USE OF HNF4ALPHA FOR TREATMENT OF HUMAN MALIGNANT SOLID TUMORS THROUGH INDUCTION-DIFFERENTIATION THERAPY

(75) Inventors: Weifen Xie, Shanghai (CN); Chuan Yin, Shanghai (CN); Yong Lin, Shanghai (CN); Yuexiang Chen, Shanghai (CN); Xin Zeng, Shanghai (CN)

(73) Assignee: SECOND MILITARY MEDICAL UNIVERSITY, Shanghai (CN)

(21) Appl. No.: 12/921,369

(22) PCT Filed: Apr. 30, 2009
(86) PCT No.: PCT/CN09/71590

§ 371 Date: Nov. 24, 2010

(30) Foreign Application Priority Data
Mar. 4, 2008 (CN) 200810034200.3

(51) Int. Cl. A61K 38/16; A61K 31/7088; C07K 14/705; C07H 21/04; A61P 35/00

(52) U.S. Cl. 514/19.5; 514/19.3; 514/44 R; 530/350; 536/23.5

(57) ABSTRACT

Use of hepatocyte nuclear factor 4α (HNF4α) for the treatment of human malignant solid tumors through induction-differentiation therapy is provided.

### TECHNOLOGICAL FIELD

The present invention relates to molecular biology, cell biology and medicine. In particular, the present invention relates to the method and application of treating solid tumors by using Hepatocyte Nuclear Factor 4α (HNF4α) to induce the differentiation of human malignant solid tumors.

### TECHNICAL BACKGROUND

Differentiation inducing treatment of tumor or differentiation therapy is the important breakthrough in clinical oncology treatment in the last 20 years. Differentiation therapy is to restore the normal cell phenotype and function and to inhibit tumor cell proliferation by inducing the differentiation to prompt the differentiation of tumor cells to mature phase. Differentiation therapy has broken the irreversible traditional understanding of the tumor development and strongly pushed the development of the whole field of cancer research.

Chinese scholars have ever used firstly the differentiation therapy of all-trans retinoic acid to treat acute promyelocytic leukemia, and have achieved good results. However, although the differentiation therapy of leukemia has made great progress, the differentiation therapy of malignant solid tumors is still a difficult problem in areas of cancer therapy research. To date, the differentiation therapies of human malignant solid tumor such as liver cancer, gastric cancer, colon cancer, kidney cancer and pancreatic cancer and others have not yet been reported clearly. So far, results have shown that all-trans retinoic acid has no obvious effect on the differentiation of malignant solid tumors.

For tumor differentiation therapy, it is difficult to select the appropriate drugs or related substances to carry out the specific targeting regulation. In some studies, drugs or proteins to regulate in vitro the differentiation status of solid tumor were used, but the effect was limited. Most in vivo experiments have suggested that the above methods play a certain role in the induction of apoptosis of tumor cells and the promotion of tumor tissue necrosis, but it is difficult to significantly regulate or reverse the poor differentiation extent of solid tumors.

It is very difficult to screen effective reagent of tumor differentiation induction from many candidate substances, and no enough effect has been proven. Therefore, choosing the key protein, molecules and genes closely related to differentiation induction of tumor cells to carry out the specific targeting regulation is one of the core problems of tumor differentiation therapy.

In recent years, the unceasingly deepening of the human genome project study creates condition for people to use genetic engineering measures to control and even change the important gene expression of cells to change the phenotypes, differentiation state and biological functions. However, although some materials or genes have been confirmed to have the capability to improve some biological characteristics in vitro of tumor cells (such as decreasing of the proliferation and colony formation capacity and up-regulating the genes expression in normal cells), some substances can even be proved to reduce the tumor formation of cancer cells in vivo of animals, it is often found that these substances have an impact on normal cells (side effects), and can not specifically induce the in vivo solid tumor differentiation. The inventors have studied the regulation effects of all-trans retinoic acid, somatostatin, tumor necrosis factor, and substances such as arsenic trioxide on in vitro differentiation of hepatocellular carcinoma cell lines, no drugs or proteins with clear differentiation therapy effects can be screened, while the in vivo study also shows that these substances can not effectively induce the differentiation of solid tumors.

Therefore, it is urgently needed in the art to develop specific proteins or genes which closely relate to the differentiation induction of malignant solid tumor cells and which can be used as targets for specific regulation, so as to effectively induce differentiation of solid tumors.

### SUMMARY OF INVENTION

The purpose of the present invention is to provide a specific gene closely related to the differentiation induction of malignant solid tumor cells, which is HNF4α gene, and its encoded product HNF4α protein, as well as the therapeutic application of HNF4α gene/protein in the differentiation induction of solid tumors.

The other purpose of the present invention is to provide a method of treatment of tumor by the differentiation induction of malignant tumor through HNF4α gene/protein.

In the first aspect of the present invention, it provides a use of Hepatocyte Nuclear Factor 4α (HNF4α) gene and/or protein in the preparation of differentiation induction agent or composition for inducing the differentiation of malignant solid tumor cells.

In another preferred embodiment, the composition is pharmaceutical composition.

In another preferred embodiment, the pharmaceutical composition comprises (a) HNF4α protein, HNF4α coding sequence or expression vector containing said coding sequence and (b) a pharmaceutically acceptable carrier or excipient.

In another preferred embodiment, the expression vector comprises viral vector and non-viral vector. Preferably, said non-viral vector is liposome.

In another preferred embodiment, the solid tumor is selected from the group consisting of liver cancer, gastric cancer, colon cancer, pancreatic cancer, lung cancer, prostate cancer and genital tumor.

In another preferred embodiment, the pharmaceutical composition is further used to suppress the in vivo formation of solid tumors.

In another preferred embodiment, the HNF4α is human HNF4α.

In another preferred embodiment, the formulation of pharmaceutical compositions is injection.

In another preferred embodiment, the pharmaceutical composition further comprises a chemotherapeutic agent.

In the second aspect of the present invention, it provides a method for inducing or improving the differentiation of solid tumor in mammal, which comprises a step of administrating HNF4α protein, its coding sequence or expression vector containing said coding sequences to a mammal subject in need of.

In another preferred embodiment, said mammal animal is human.

### DESCRIPTION OF DRAWINGS

FIG. 1 shows RT-PCR detection on the expression of HNF4α gene and the related function genes of the hepatocytes in the human liver tumor cell lines.

FIG. 2 shows HNF4α cDNA fragment obtained by RT-PCR.

FIG. 3 shows Restriction enzyme digestion identification of shuttle plasmid pAdTrack-CMV-HNF4α Bgl II and EcoR V obtained from the in vitro ligation.

FIG. 4 shows Pac I restriction endonuclease identification of recombinant adenovirus plasmid pAdHNF4α.

FIG. 5 shows restriction endonuclease identification of recombinant adenovirus plasmid pAdHNF4α.

FIG. 6 shows GFP expression 3 days after the HepG2 (A, B) and Hep3B (C, D) cells were infected by AdHNF4α.

FIG. 7 shows Western blot detection on HNF4α protein expression 3 days after the liver tumor cells were infected by AdHNF4α.

FIG. 8 shows quantitative analysis of HNF4α protein expression 3 days after the liver tumor cells were infected by AdHNF4α.

FIG. 9 shows RT-PCR detection on the quantitative analysis of mRNA expression of HNF4α gene and the related function genes of the hepatocytes in the human liver tumor cell lines.

FIG. 10 shows ammonia metabolism 3 days after the liver tumor cells were infected by AdHNF4α.

FIG. 11 and FIG. 12 show the expression determination of CD133 after the liver tumor cells were infected by AdHNF4α.

FIG. 13 and FIG. 14 show the Effects of exogenous HNF4α transfer on colony formation of human solid tumor cell.

FIG. 15 shows ICG absorption staining determination after the liver tumor cells were infected by AdHNF4α.

FIG. 16 shows tumor formation experiment of in vivo inoculation after the liver tumor cells were infected by AdHNF4α.

FIG. 17 shows the differentiation inducing therapy of the experimental liver tumor model with HNF4α gene.

### DETAILED DESCRIPTION

After extensive and intensive researches, the inventors have found a key gene/protein, i.e. HNF4α gene/protein, for the first time, which can effectively induce or promote the differentiation of solid tumor to normal cells. Experiments showed that, HNF4α impact on not only tumor cell apoptosis, but also the differentiation of solid tumor cells by generating induction or promotion effects (cell morphological changes, significant up-regulation expression of functional genes related to liver cells and in vivo tumor prevention, intervention and treatment effects). Therefore, HNF4α, the key gene/protein which has been confirmed for the first time to effectively induce or promote the differentiation of solid tumor to normal cells in vivo, has a potential application prospect. The inventors finished the present invention based on the above discoveries.

As used herein, the term “gene/protein” means gene and/or protein.

As used herein, the terms “HNF4α Protein”, “HNF4α Polypeptide”, “the Polypeptide of the invention” and “the Protein of the present invention” are exchangeable, referring to Hepatocyte Nuclear Factor 4α protein. It includes the HNF4α with or without the starting Met. In the narrow sense, said term refers to human HNF4α; and in the broad sense, said term includes not only human HNF4α but also other mammal HNF4α, especially quadrumana HNF4α, such as ape or monkey HNF4α. This term also includes active fragments, active derivatives and analogs of HNF4α protein.

The polypeptide of invention may be a recombinant, natural, or synthetic polypeptide, preferably a recombinant polypeptide. The polypeptide of invention may be a purified natural product or a chemically synthetic product. Alternatively, it may be produced from prokaryotic or eukaryotic hosts, such as bacteria, yeast, higher plant, insect, and mammalian cells, using recombinant techniques. According to the host used in the recombinant production, the polypeptide may be glycosylated or non-glycosylated. The polypeptide may or may not comprise the starting Met residue.

As used in the invention, the terms “fragment “,” derivative “and” analogue” mean the polypeptide that essentially retains the same biological functions or activity of natural HNF4α protein. The fragment, derivative or analogue of the polypeptide may be (i) one in which one or more of the amino acid residues are substituted with a conserved or non-conserved amino acid residue (preferably a conserved amino acid residue), or (ii) one in which one or more of the amino acid residues include a substituent group, or (iii) one in which the mature polypeptide is fused with another compound, such as a compound to increase the half-life of the polypeptide (for example, polyethylene glycol), or (iv) one in which the additional amino acids are fused to mature polypeptide, such as a leader or secretary sequence or a sequence used for purifying polypeptide or proprotein, e.g., a fusion protein formed with IgC fragment. Such fragments, derivatives and analogs are known to the artisans based on the teachings herein.

In the present invention, the term “human HNF4α polypeptide” means a polypeptide comprising the wild-type HNF4α amino acid sequence. The term also comprises the variants which have the same function of human HNF4α, i.e., inducing the differentiation of solid tumors. These variants include, but are not limited to, deletions, insertions and/or substitutions of several amino acids (typically 1-50, preferably 1-30, more preferably 1-20, most preferably 1-10), and addition of one or more amino acids (typically less than 20, preferably less than 10, more preferably less than 5) at C-terminal and/or N-terminal. E.g., the protein functions are usually unchanged when an amino residue is substituted by a similar or analogous one. Further, the addition of one or several amino acids at C-terminal and/or N-terminal usually does not change the protein function. The term also includes the active fragments and derivatives of human HNF4α protein.

The invention also provides the analogues of human HNF4α polypeptide. Analogues can differ from naturally occurring human HNF4α polypeptide by amino acid sequence differences or by modifications which do not affect the sequence, or by both. Also included are analogues which include residues other than those naturally occurring L-amino acids (e.g., D-amino acids) or non-naturally occurring or synthetic amino acids (e.g., beta- or gamma-amino acids).

Modifications (which do not normally alter primary sequence) include in vivo or in vitro chemical derivation of polypeptides, e.g., acetylation, or carboxylation. Also included are modifications of glycosylation, e.g., those made by modifying the glycosylation patterns of a polypeptide during its synthesis and processing or in the further processing steps, e.g., by exposing the polypeptide to glycosylation enzymes (e.g., mammalian glycosylating or deglycosylating enzymes). Also included are sequences having phosphorylated amino acid residues, e.g., phosphotyrosine, phosphoserine, phosphothronine, as well as sequences modified to improve the resistance to proteolytic degradation or to optimize solubility properties.

In the invention, “human HNF4α conservative mutant” means a polypeptide formed by substituting at most 10, preferably at most 8, more preferably 5, and most preferably at most 3 amino acids with the amino acids having substantially the same or similar property, as compared with the wild-type amino acid sequence. Preferably, these conservative mutants are formed by the substitution according to Table 1.

TABLE 1

| Initial residue | Representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

As used herein, the terms “HNF4α gene” and “gene of the invention” are exchangeable, referring to a polynucleotide sequence which encodes HNF4α protein. The polynucleotide of invention may be in the forms of DNA and RNA. DNA includes cDNA, genomic DNA, and synthetic DNA, etc., in single strand or double strand form. A single strand DNA may be an encoding strand or non-encoding strand. The coding sequence for mature polypeptide may be identical to the wild-type coding sequence, or is a degenerate sequence. As used herein, the term “degenerate sequence” means a sequence which encodes a protein comprising the wild-type sequence and which has a nucleotide sequence different from the wild-type coding region.

The term “polynucleotide encoding the polypeptide” includes the polynucleotide encoding said polypeptide and the polynucleotide comprising additional and/or non-encoding sequence.

The invention further relates to the variants of polynucleotides which encode a polypeptide having the amino acid sequence of the HNF4α as described hereinabove, or its fragment, analogue and derivative.

The full-length human HNF4α nucleotide sequence or its fragment can be prepared by PCR amplification, recombinant method and synthetic method. For PCR amplification, one can obtain said sequences by designing primers based on the nucleotide sequence disclosed herein, especially the ORF, and using cDNA library commercially available or prepared by routine techniques in the art as a template.

Once the sequence is obtained, one can produce lots of the sequences by recombinant methods. Usually, said sequence is cloned into a vector which is then transformed into a host cell. The sequence is isolated from the amplified host cells using conventional techniques.

The invention further relates to a vector comprising the polynucleotide of invention, a genetic engineered host cell transformed with the vector or the sequence encoding human HNF4α protein, and the method for producing the human HNF4α polypeptide by recombinant techniques.

The recombinant human HNF4α polypeptides can be expressed or produced by the conventional recombinant DNA technology, using the polynucleotide sequence of invention. Generally, it comprises the following steps:

(1) transfecting or transforming the appropriate host cells with the polynucleotide encoding human HNF4α polypeptide or the vector containing the polynucleotide;

(2) culturing the host cells in an appropriate medium;

(3) isolating or purifying the protein from the medium or cells.

In the invention, the polynucleotide sequences encoding human HNF4α may be inserted into a recombinant expression vector. The term “recombinant expression vector” means a bacterial plasmid, bacteriophage, yeast plasmid, plant virus or mammalian cell virus, such as adenovirus, retrovirus or any other vehicles known in the art. Vectors suitable for use in the present invention include, but are not limited to, the T7-based expression vector for expression in bacteria (Rosenberg, et al., Gene, 56:125, 1987), and the pMSXND expression vector for expression in mammalian cells (Lee and Nathans, J. Biol. Chem., 263:3521, 1988). On the whole, any plasmid or vector can be used to construct the recombinant expression vector as long as it can replicate and is stable in the host. One important feature of expression vector is that the expression vector typically contains an origin of replication, a promoter, a marker gene as well as the translation regulatory components.

The known methods can be used to construct an expression vector containing human HNF4α DNA sequence and appropriate transcription/translation regulatory components. These methods include in vitro recombinant DNA technique, DNA synthesis technique, in vivo recombinant technique, etc (Sambroook, et al. Molecular Cloning, a Laboratory Manual, cold Spring Harbor Laboratory, New York, 1989). The DNA sequence is efficiently linked to the proper promoter in an expression vector to direct the synthesis of mRNA. The exemplary promoters are lac or trp promoter of E. coli; P_{L} promoter of λ phage; eukaryotic promoter including CMV immediate early promoter, HSV thymidine kinase promoter, early and late SV40 promoter, LTRs of retrovirus and some other known promoters which control the gene expression in the prokaryotic cells, eukaryotic cells or virus. The expression vector may further comprise a ribosome-binding site for initiating the translation, transcription terminator and the like.

The expression vector preferably comprises one or more selective marker genes to provide a phenotype for selecting the transformed host cells, e.g., the dehydrofolate reductase, neomycin resistance gene and GFP (green flurencent protein) for eukaryotic cells, as well as tetracycline or ampicillin resistance gene for E. coli.

The vector containing said DNA sequence and proper promoter or regulatory elements can be transformed into appropriate host cells to express the protein.

The “host cell” includes prokaryote, e.g., bacteria; primary eukaryote, e.g., yeast; advanced eukaryotic, e.g., mammalian cells. The representative examples are bacterial cells, e.g., E. coli, Streptomyces, fungal cells, e.g., yeast; animal cells e.g., CHO, COS or 293 cell, etc.

Recombinant transformation of host cell with the DNA might be carried out by conventional techniques known to the artisans. Where the host is prokaryotic, e.g., E, coli, the competent cells capable of DNA uptake, can be prepared from cells harvested after exponential growth phase and subsequently treated by the CaCl₂ method using known procedures. The transformation can also be carried out by electroporation. When the host is an eukaryote, transfection of DNA such as calcium phosphate co-precipitates, conventional mechanical procedures e.g., micro-injection, electroporation, or liposome-mediated transfection may be used.

The transformants are cultured conventionally to express HNF4α polypeptide. According to the used host cells, the medium for cultivation can be selected from various conventional mediums. The host cells are cultured under a condition suitable for its growth until the host cells grow to an appropriate cell density. Then, the selected promoter is induced by appropriate means (e.g., temperature shift or chemical induction) and cells are cultured for an additional period.

In the above methods, the recombinant polypeptide may be included in the cells, or expressed on the cell membrane, or secreted out. If desired, the physical, chemical and other properties can be utilized in various isolation methods to isolate and purify the recombinant protein. These methods are well-known to the artisans and include, but are not limited to conventional renaturation treatment, treatment by protein precipitant (e.g., salt precipitation), centrifugation, cell lysis by osmosis, sonication, supercentrifugation, molecular sieve chromatography or gel chromatography, adsorption chromatography, ion exchange chromatography, HPLC, and any other liquid chromatography, and the combination thereof.

Recombinant HNF4α polypeptide can be directly used as differentiation inducing agent to induce or prompt the differentiation of malignant solid tumors. Additionally, polynucleotides encoding HNF4α protein or vectors containing HNF4α encoding sequences may also have the same therapeutic effect.

The methods to introducing polynucleotides into tissues or cells include injecting polynucleotide directly into in vivo tissues, or firstly importing polynucleotide in vitro into the cells by vectors (such as virus, bacteriophage or plasmid) and then transplanting the cells in corpora, etc.

Recombinant gene therapy vectors (such as virus vectors) can be designed to express wild-type HNF4α protein so as to increase the quantity and activity of HNF4α protein in the solid tumor. The expression vectors derived from virus, such as retrovirus, adenovirus, adeno-associated virus, herpes simplex virus, parvovirus, and so on, can be used to introduce the HNF4α gene into the cells. The methods for constructing a recombinant virus vector harboring HNF4α gene are described in the literature (Sambrook, et al.). In addition, the recombinant HNF4α gene can be packed into liposome and then transferred into the cells.

When HNF4α protein, HNF4α polynucleotide and its vectors mentioned in the present invention, are administrated to mammal subjects (such as human), they can induce or prompt the differentiation of malignant solid tumors. Usually, these substances are formulated with a non-toxic, inert and pharmaceutically acceptable aqueous carrier. The pH typically is about 5-8, preferably 6-8, although pH may alter according to the property of the formulated substances and the diseases to be treated. The formulated pharmaceutical composition is administrated in conventional routes including, but not limited to, intratumoral, intramuscular, intravenous, subcutaneous, intradermal or topical administration.

Pharmaceutical compositions of this invention can be directly used to induce the differentiation of solid tumors (therapy). The representative examples include but are not limited to liver cancer, gastic cancer, colon cancer, lung cancer, pancreatic cancer, renal cancer, prostate cancer and genital tumors.

The human HNF4α gene/protein or drug composition of this invention can be administrated in combination with other medicaments, such as cisplatin, TNF, etc.

The invention also provides a pharmaceutical composition comprising safe and effective amount (e.g. 0.0001-90 wt %) of human HNF4α protein in combination with a pharmaceutically acceptable carrier. Such a carrier includes but is not limited to saline, buffer solution, glucose, water, glycerin, ethanol, or the combination thereof. The pharmaceutical formulation should be suitable for delivery method. The pharmaceutical composition may be in the form of injections which are made by conventional methods, using physiological saline or other aqueous solution containing glucose or auxiliary substances. The pharmaceutical compositions in the form of tablet or capsule may be prepared by routine methods. The pharmaceutical compositions, e.g., injections, solutions, tablets, and capsules, should be manufactured under sterile conditions. The pharmaceutical composition of the present invention may contain other therapeutic agents such as chemotherapeutic agents.

When using pharmaceutical composition, the safe and effective amount of the HNF4α protein, HNF4α polynucleotide or the vector are administrated to mammals. Typically, the safe and effective amount is at least about 1 μg/kg body weight and less than about 10 mg/kg body weight in most cases, and preferably about 10 ug-1 mg/kg body weight. Certainly, the precise amount depends upon various factors, such as delivery methods, the subject health, etc., and is within the judgment of the skilled clinician.

The present invention also provides a method for inducing and/or prompting the differentiation of malignant solid tumors, which comprises administrating HNF4α protein, HNF4α polynucleotide or expression vector to a mammal subject (such as human) in need of, thereby in vivo inducing and/or prompting the differentiation of malignant solid tumors

The present invention also provides a gene therapy to treat tumor cells (especially for the malignant solid tumors), in which HNF4α gene is transferred into tumor cells to express HNF4α, wherein the transfer methods include plasmid transfection, adenovirus or adeno-associated virus mediation.

The main advantages of the present invention include:

(a) The differentiation therapy of malignant solid tumor was confirmed for the first time by using a variety of genetic engineering methods.

(b) The important transcription factor HNF4α was screened and the regulation effects thereof on the differentiation of malignant solid tumors was confirmed.

(c) We confirmed the feasibility and potential significance to clinical studies in vivo and in vitro of the differentiation therapy for malignant solid tumors.

The invention is further illustrated by the following examples. These examples are only intended to illustrate the invention, but not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### Example 1

### RT-PCR Detection on the Expression of HNF4α Gene and the Related Function Genes of the Hepatocytes in the Human Liver Tumor Cell Lines

1. The commercially available and conventional liver tumor cell lines Huh-7, Hep3B, HepG2 were inoculated onto the six-well plate with the concentration of 8×10⁵ cells/dish, and were cultured in the fresh medium containing 10% fetal bovine serum. On the next day, the RNA of the cells was extracted, and the OD260 value was determined by spectrophotometer, with the working concentrations of 1 μg/μl and 0.1 μg/μl. The integrity of RNA was tested by 1% agarose gel electrophoresis.

2. RT-PCR: 4 μg of RNA, 2 μl of random primer, DEPC were taken, and water was added to total volume of 33 μl. The solution was set at 70° C. for 5 min, 0° C. for 5 min. Then, 10 μl of 5× Buffer, 3 μl of dNTP, 2 μl of RNA reverse transcriptase and 2 μl of RNA enzyme inhibitor were added and mixed, and were set at 37° C. for 1.5 h. The reverse transcription products were obtained. 1 μl diluted reverse transcription product was taken as a template for PCR amplification. The gene primer sequences, reaction conditions, and the reaction system for detection were shown in Tables 2 and 3. RT-PCR products of each group were identified by using 1.5% agarose gel electrophoresis, and the images were scanned. The commercially available Multy-Analasyst image analysis software was used for optical scanning and sequencing analysis.

TABLE 2
Reaction system, human primer sequences and reaction conditions

| Components | Volume (μl) | |
|---|---|---|
| Sense primer | 0.3 | μl |
| Antisense primer | 0.3 | μl |
| Reverse transcription products | 1 | μl |
| Taq enzyme | 0.2 | μl |
| 10 × Taq buffer | 1.5 | μl |
| dNTP | 0.4 | μl |
| ddH₂O | 11.3 | μl |

TABLE 3
Human primer sequences and reaction conditions

| | | SEQ ID | | Annealing | |
|---|---|---|---|---|---|
| Gene | Primer Sequence | NO: | Product | Temp. | Cycles |
| HNF4α | Sense: 5′-TTGAAAATGTGCAGGTGTTGAC-3′ | 1 | 429 bp | 55° C. | 29 |
| | antisense: 5′-CAGAGATGGGAGAGGTGATCTG-3′ | 2 | | | |
| APOCIII | Sense: 5′-GGGTACTCCTTGTTGTTGC-3 | 3 | 250 bp | 55° C. | 31 |
| | antisense: 5′-AAATCCCAGAACTCAGAGAAC-3 | 4 | | | |
| G-6-P | Sense: 5′-GGCTCCATGACTGTGGGATC-3′ | 5 | 475 bp | 49° C. | 32 |
| | antisense: 5′-TTCAGCTGCACAGCCCAGAA-3′ | 6 | | | |
| ALB | Sense: 5′-AGCCTAAGGCAGCTTGACTT-3′ | 7 | 1212 bp | 55° C. | 29 |
| | antisense: 5′-CTCGATGAACTTCGGGATGA-3′ | 8 | | | |
| GS | Sense: 5′-CCTGCTTGTATGCTGGAGTC-3′ | 9 | 396 bp | 55° C. | 35 |
| | antisense: 5′-GAAAAGTCGTTGATGTTGGA-3′ | 10 | | | |
| CYP1a2 | Sense: 5′-CTGGCCTCTGCCATCTTCTG-3′ | 11 | 464 bp | 49° C. | 35 |
| | antisense: 5′-TTAGCCTCCTTGCTCACATGC-3′ | 12 | | | |
| PEPCK | Sense: 5′-GTGTCCCTCTAGTCTATGAAGC-3′ | 13 | 485 bp | 49° C. | 35 |
| | antisense: 5′-ATTGACTTGATCCTCCAGATAC-3′ | 14 | | | |
| TTR | Sense: 5′-GCGGGACTGGTATTTGTGTCTG-3′ | 15 | 398 bp | 55° C. | 27 |
| | antisense: 5′-TTAGTGACGACAGCCGTGGTG-3′ | 16 | | | |
| AFP | Sense: 5′-AGCTTGGTGGTGGATGAAAC-3′ | 17 | 248 bp | 55° C. | 35 |
| | antisense: 5′-CCCTCTTCAGCAAAGCAGAC-3′ | 18 | | | |
| β-actin | Sense: 5′-CATCCTGCGTCTGGACCT-3′ | 19 | 499 bp | 55° C. | 25 |
| | antisense: 5′-GTACTTGCGCTCAGGAGGAG-3′ | 20 | | | |

Hepatocyte nuclear factor 4α (HNF4α); glucose -6- phosphatase (G-6-P); albumin (ALB); Glutamine synthetase (GS); family of cytochrome P450 1a2 (CYP1a2); phosphoenolpyruvate carboxykinase (PEPCK); thyroid hormone binding protein (transthyretin, TTR); alpha fetoprotein (AFP); apolipoprotein CIII (APOCIII)

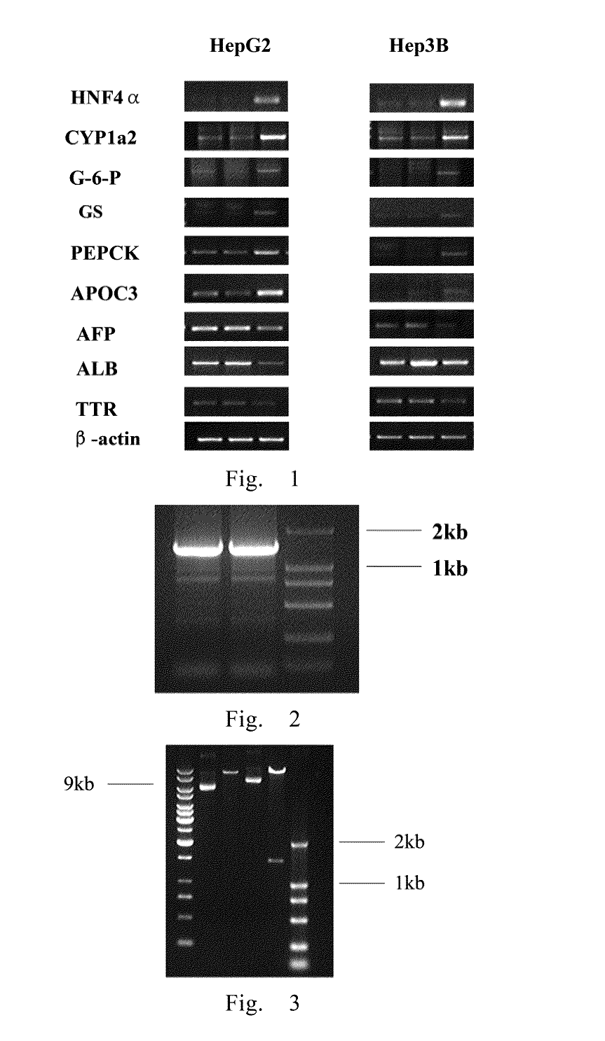

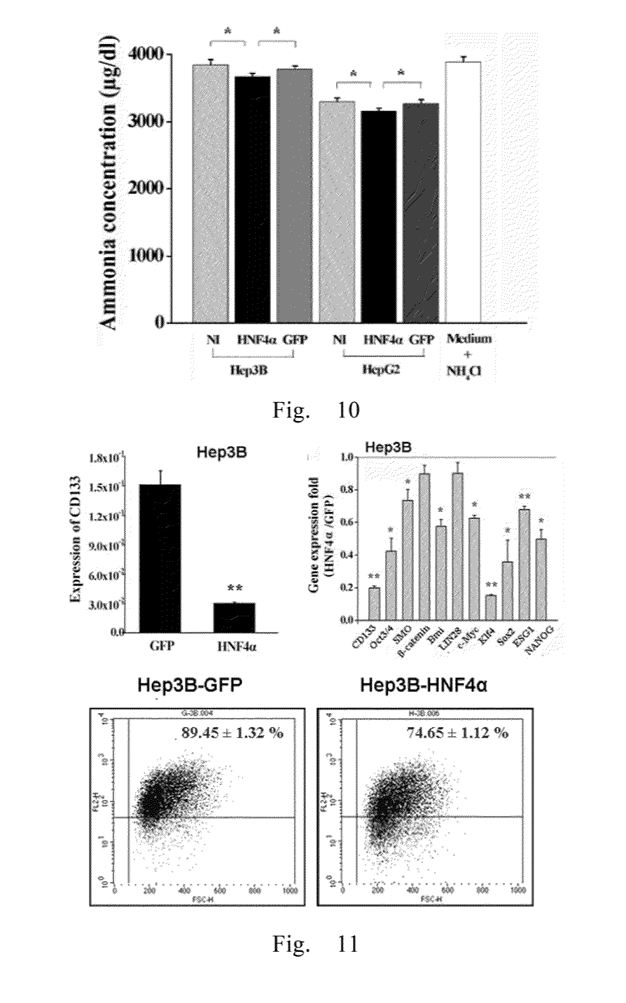

The results showed that the expression of HNF4α in liver tumor cell lines Huh-7, Hep3B and HepG2 were significantly reduced, and HNF4α gene expression had a positive correlation with the important function gene expressions related to liver cell (FIG. 1 and FIG. 9).

### Example 2

### Preparation of Replication-Defective Recombinant Adenovirus for HNF4α Expression

1. Obtaining HNF4α 1425 bp cDNA fragment: The primers were designed and synthesized according to HNF4α cDNA sequences. Sense primer (Bgl II restriction enzyme cutting site was added at 5′ end): 5′-CCG AGA TCT AGA ATG CGA CTC TCC AAA ACC-3′ (SEQ ID NO: 21); antisense primer (EcoRV restriction enzyme cutting site was added at 5′ end): 5′-CGC GAT ATC GGC TTG CTA GAT AAC TTC CTG CT-3′(SEQ ID NO: 22).

HNF4α cDNA fragment was amplified by PCR, and the product was isolated using 1% agarose gel electrophoresis. The fragment size was identified and the gel block was cut and transferred into Eppendorf tube. The gel was weighed. 200 ml NT solution/100 mg gel was added into the Eppendorf tube, the tube was heated at 50° C. for 5-10 mins until the gel melt. The liquid was flown through columns, centrifugated 1 min at 13,000 rpm, and 600 μl NT3 buffer solution was added, centrifugated 2 mins at 13,000 rpm. 30 μl double-distilled water was flown though columns to elute DNA fragment. After placing for 1 min, it was centrifugated 1 min at 13,000 rpm. The eluent was carefully pipetted into clean Eppendorf tube. OD260 value was measured with spectral photometer and fragment size was identified using 1.5% agarose gel electrophoresis (FIG. 2).

| Components | Volume (μl) | |
|---|---|---|
| Sense primer | 5 | μl |
| Antisense primer | 5 | μl |
| Normal human liver cell cDNA | 2 | μl |
| pfu enzyme | 2 | μl |
| 10 × pfu buffer solution | 10 | μl |
| dNTP | 10 | μl |
| ddH₂O | 66 | μl |

Reaction conditions: 94° C. 30 s, 60° C. 30 s, 72° C. 90 s, 35 cycles.

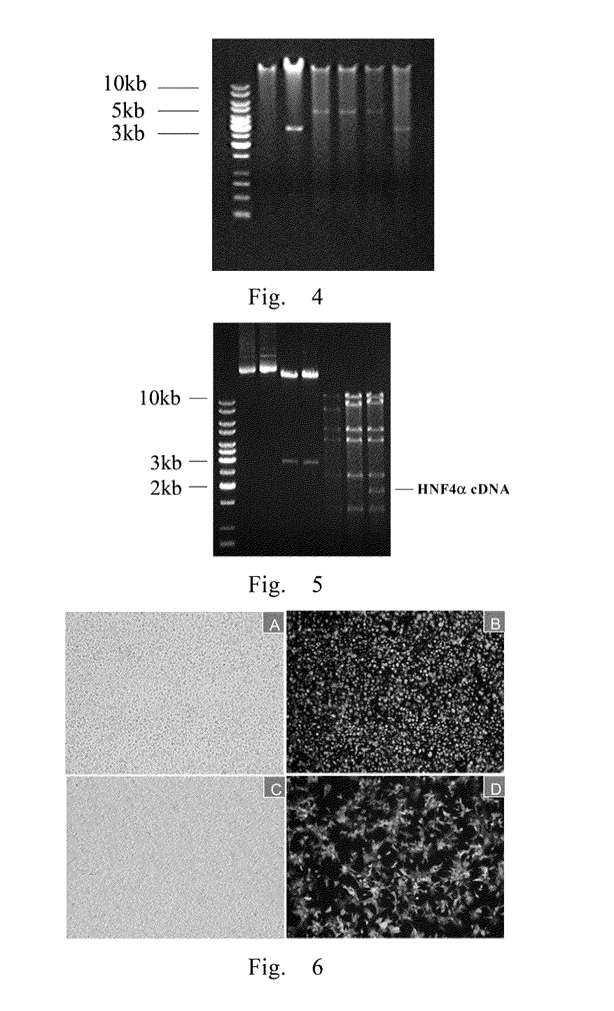

2. Establishing adenovirus plasmid pAdHNF4α for expression of HNF4α: After using EcoRV and Bgl II enzyme to digest shuttle plasmid pAdTrack-CMV (Purchase from Howard Hughes Medical Institute (HHMI), USA) and purifying, 0.1 μl plasmid pAdTrack-CMV, 0.4 μg HNF4α cDNA, 10×T4 buffer solution 2 μl, T4 DNA ligase 1 μl (2 U) and ddH₂O were taken (total volume was 20 μl). The mixture was ligated overnight at 16° C. The ligated products were used to transfer the conventional competent E. coli DH5α. LB medium containing kanamycin was spread on the plate, placed at constant temperature 37° C. overnight; and colonization of single colony was chosen. The colonies which could produce amplified HNF4α cDNA fragment were extracted using Qiagen-tip 100 kit. Plasmid pAdTrack-CMV-HNF4α was obtained and identified. pAdTrack-CMV-HNF4α was cleaved with Pme I enzyme to make it linear. 0.4 μg linear pAdTrack-CMV-HNF4α and 0.1 μg superhelix pAdEasy-1 plasmid were separately taken and used to co-transfer 20 μl competent BJ5183 bacteria by electroporation (2,000 V, 200 Ohms, 25μ FD). The transformants were screened out using kanamycin-containing LB medium plate, and viral plasmid pAdHNF4α was chosen and identified. (FIGS. 4 and 5)

3. Packing and amplifying adenovirus AdHNF4α: The method is as follows: Recover regular 293 cells, inoculate on 10-cm tissue plate at 4.8×10⁶/plate, add DMEM 37° C. and culture in 5% CO₂ incubator; cell density reaches to 60%-80% after 24 hours of growth. pAdHNF4α is cleaved with Pac I enzyme and mixed with 250 μl serum-free DMEM culture solution to prepare solution A. 20 μl Lipofectamin is taken and 250 μl serum-free DMEM culture solution is added, thereby forming solution B. Fully mix solutions A and B, place the mixture at room temperature for 30 mins, then the 293 cells are added into for transfection. Change culture solution 4 hours later. 7 days later, 293 cells and its supernatant fluid are collected, freezed and thawn repeatedly in liquid nitrogen and 37° C. water bath for 4 times, centrifugated 5 mins at 5,000 rpm. Virus supernatant fluid is collected and used to again to infect 293 cells for amplification. The virus is collected 2-3 days later. Repeat the steps of infection and collection, separately pack the virus supernatant fluid which is finally collected, and measure the titer of virus supernatant fluid. The adenovirus AdHNF4α with titer of 1×10¹⁰ efu/ml was finally obtained, and stored at −80° C. for use.

### Example 3

### Using RT-PCR and Western Blot to Test the Expression of HNF4α in Human Liver Tumor Cell Line Infected by the AdHNF4α

1. Human hepatoma cell lines HepG2 and Hep3B were inoculated onto the six-well plate with a concentration of 5×10⁵ cells/dish. The cells were infected by the virus AdHNF4α with MOI 40 and 100, respectively. After 24 hours, the medium were replaced by fresh DMEM culture solution containing 10% FBS, and the expression of GFP was observed after 3 days (FIG. 6). The total RNA was extracted with Trizol kit, and the reverse transcription reaction was conducted for 2 hours. 1 μl diluted reverse transcription product was used as template to carry out the amplification reaction of HNF4α PCR and the reaction conditions were the same as above. At the same time, the PCR reaction of the β-actin was carried out in the same reaction conditions an used as the internal control. The reaction system was as follows.

| Components | Volume (μl) | |
|---|---|---|
| Sense primer | 0.3 | μl |
| Antisense primer | 0.3 | μl |
| Reverse transcription products | 1 | μl |
| Taq enzyme | 0.2 | μl |
| 10 × Taq buffer solution | 1.5 | μl |
| dNTP | 0.4 | μl |
| ddH₂O | 11.3 | μl |

Reaction conditions were 95° C. 30 s, 55° C. 30 s, 72° C. 90 s, 27 cycles.

The RT-PCR products were identified using 1.5% agarose gel electrophoresis, and the images were scanned. The Multy-Analasyst image analysis software was used for optical scanning and sequencing analysis.

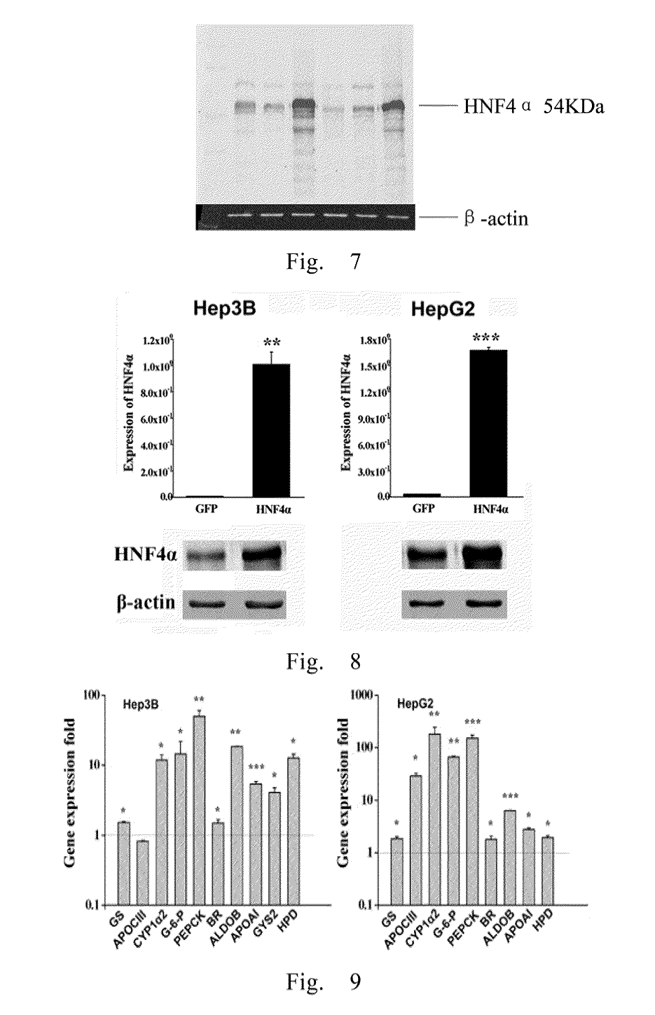

The results showed that the expression of HNF4α mRNA in tumor cells increased significantly after the AdHNF4α infection of human liver tumor cell lines (FIGS. 7 and 8).

2. HepG2 and Hep3B were respectively infected by AdHNF4α, the whole cell proteins were collected from the cell lysate. After standard quantitative analysis of protein, 10 μg of protein was taken an run in 10% SDS-PAGE electrophoresis, thereby to separating the proteins. The polyvinylidene fluoride membrane (PVDF membrane) was washed by ddH₂O, and the running gel, PVDF membrane, filter paper were placed in the Transferring Buffer for balance, then were placed in the electric transfer tank with 18 V of voltage for 40 min. The membrane was blocked with 20 ml of 5% BSA/PBST for 2 hours at room temperature and incubated with HNF4α multiple antibody (1:500) at 4° C. overnight. On the next day, after the PBST washing, it was incubated with the donkey anti-goat fluorescent secondary antibodies (1:2000) for 30 min at room temperature. After the PBST washing for twice, the fluorescence was measured using the Odyssey infrared laser imaging system and the grayscale scanning was carried out.

The results showed that the protein expression of HNF4α after infecting the human tumor cell lines by AdHNF4α was increased 3.4 times (HepG2) and 5.2 times (Hep3B), respectively (FIGS. 7 and 8).

### Example 4

### Effects of Exogenous HNF4α on the Biological Characteristics of Human Hepatoma Cells

1. RT-PCR Detection on Function Gene Expression Related to Liver Cell:

The normal human liver tumor cell lines HepG2 and Hep3B were inoculated onto the 6-well plate at a concentration of 5×10⁵ cells/dish, and the cells were infected by the virus AdHNF4α with MOI 40 and 100, respectively. After 24 h, the medium was replaced with fresh DMEM culture solution containing 10% FBS. After 3 days, the GFP expression was observed. The total RNA was extracted by Trizol kit, and the reverse transcription reaction was carried out for 2 h. 1 μl diluted reverse transcription product was used as template for PCR amplification. The primer sequence for gene detection and the reaction conditions were the same as those in Example 1, and the reaction system was as follows.

| Components | Volume (μl) | |
|---|---|---|
| Sense primer | 0.3 | μl |
| Antisense primer | 0.3 | μl |
| Reverse transcription products | 1 | μl |
| Taq enzyme | 0.2 | μl |
| 10 × Taq buffer solution | 1.5 | μl |
| dNTP | 0.4 | μl |
| ddH₂O | 11.3 | μl |

RT-PCR products of each group were identified using 1.5% agarose gel electrophoresis, and the images were scanned. The Multy-Analasyst image analysis software was used for optical scanning and sequencing analysis.

The results showed that, compared with the control group, the expression of functional gene related to the liver cell in the AdHNF4α infection group was significantly up-regulated, wherein G-6-P mRNA expression was increased about 50-folds, while no significant change was observed for expression of ALB and TTR, and AFP expression was significantly reduced (Table 3-1).

TABLE 3-1
semi-quantitative analysis of the expression of related functional genes
in liver cells after HNF4α gene transfection

| | Cell lines | | | |
|---|---|---|---|---|
| | Hep3B | | HepG2 | |
| | Optical density ratio | | Optical density ratio | |
| | AdHNF4α Group | | AdHNF4α Group | |
| Gene | AdGFP Group | P value | AdGFP Group | P value |
| GS | 2.9-fold | P < 0.01 | 12.6-fold | P < 0.01 |
| PEPCK | 16.7-fold | P < 0.01 | 5.0-fold | P < 0.01 |
| G-6-P | 52.3-fold | P < 0.01 | 2.3-fold | P < 0.05 |
| CYP1a | 6.1-fold | P < 0.01 | 18.6-fold | P < 0.01 |
| APOC□ | 4.9-fold | P < 0.01 | 5.7-fold | P < 0.01 |
| AFP | 9.3% | P < 0.01 | 24.7% | P < 0.01 |

2. Flow Cytometry Determination of Human Liver Tumor Cell Apoptosis:

The normal hepatoma cell lines HepG2 and Hep3B were inoculated onto the six-well plate with a concentration of 5×10⁵ cells/dish, and the cells were infected by the virus AdHNF4α with MOI 40 and 100, respectively. After 24 hrs, the medium were replaced with fresh DMEM culture solution containing 10% FBS. The cells were collected on the third day. The cell apoptosis rate was determined using the EPICS XL flow cytometer (Coulter) and the statistical analysis was carried out. Two dishes were replicated for each group. The experiment was repeated for three times.

The results showed that the apoptosis rate was affected to a certain extent after the expression of HNF4α was up-regulated in hepatoma cells.

3. Flow Cytometry Determination of Human Liver Tumor Cell Cycle Changes.

The hepatoma cell lines HepG2 and Hep3B were inoculated onto the six-well plate with a concentration of 5×10⁵ cells/dish, and the cells were infected by the virus AdHNF4α with MOI 40 and 100, respectively. After 24 hrs, the medium was replaced with fresh DMEM culture solution containing 10% FBS. The cells were collected on the third day. The cell cycle changes were determined using the EPICS flow cytometer (Coulter) and the statistical analysis was carried out.

The results showed that HepG2 cells in S phase were decreased after 72 h and 96 h of virus infection.

4. Ammonia Concentration Detection in Cell Supernatant Using Conventional Kit

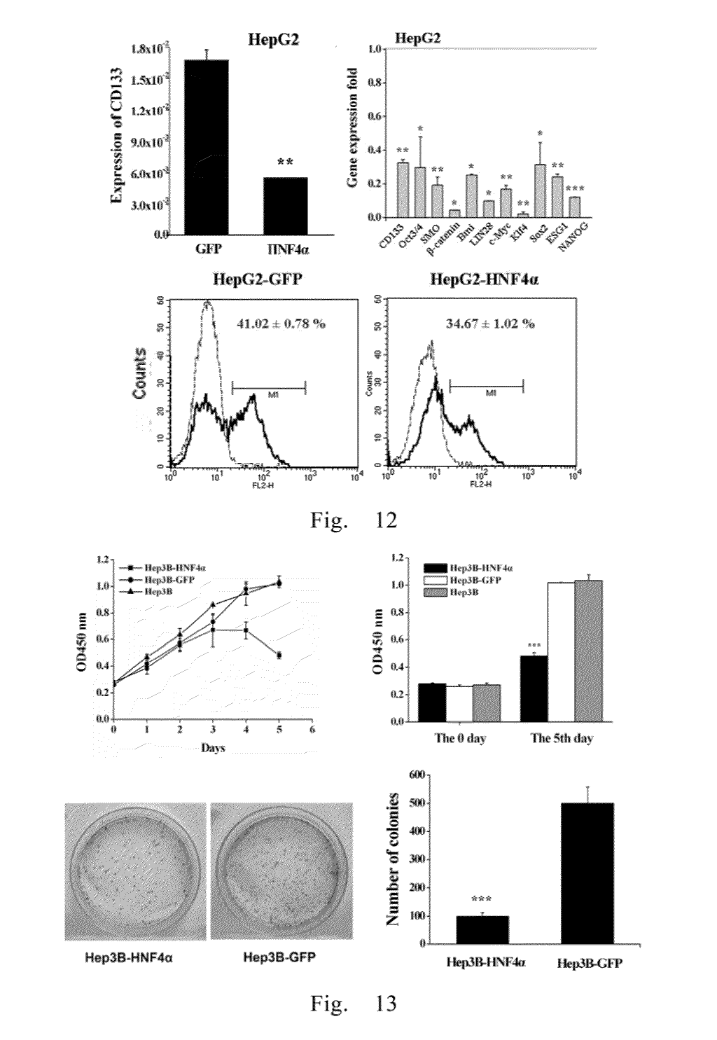

The results showed that, compared with the control group (no virus or empty virus), the ammonia metabolism capability in the experimental group was increased significantly (FIG. 10).

### Example 5

### Effects of Exogenous HNF4α on Proliferation of Human Solid Tumor Cells

Human liver tumor cell lines, gastric cancer cell lines and colon cancer cell lines were independently inoculated onto the 96-well plates with a concentration of 5×10³ cells/well. After 24 hrs, the cells were infected by the virus AdHNF4α. Then, on a daily basis, the absorbance at the wavelength of 450 nm was measured with CCK8 reagent to determine the number of active cells.

The results showed that HNF4α expression had a significant inhibition on solid tumor cell proliferation, which began firstly from the third day after virus infection. At that time, the tumor cell proliferation was found to decrease in AdHNF4α infection group, and to decrease significantly on the fifth day, with an inhibition rate up to 50%-68%. The study also showed that, with the increase of virus infection titer, the inhibition effect from HNF4α up-regulated expression on the proliferation of some solid tumor cells was time-dependent and dose-dependent.

### Example 6

### Effects of Exogenous HNF4α on the CD133 Expression of Human Hepatoma Cells

1. Human hepatoma cell lines HepG2 and Hep3B were inoculated onto the six-well plate with a concentration of 5×10⁵ cells/dish, and the cells were infected by the virus AdHNF4α with MOI 40 and 100, respectively. After 24 hrs, the medium was replaced with fresh DMEM culture solution containing 10% FBS. After three days, the cells were collected, and the CD133/1-PE (Miltenyi Biotec, Auburn, Calif.) was used as primary antibody for incubation, and the proportion of CD133+ cells was determined by flow cytometry.

The results showed that the proportion of CD133+ cells in liver tumor cells decreased significantly after the AdHNF4α infection (FIGS. 11 and 12).

CD133+ is a specific marker of cancer stem cells. The significant decrease of CD133+ cell proportion demonstrated that AdHNF4α promoted and induced the differentiation of stem cells so that the proportion was decrease significantly. In addition, for all-trans retinoic acid or arsenic trioxide, no significant differentiation induction effect on tumor stem cells was observed (data not shown).

### Example 7

### Effects of Exogenous HNF4α on Colony Formation of Human Solid Tumor Cells

Human liver tumor cell lines, gastric cancer cell lines and colon cancer cell lines were independently inoculated into the 35 mm Petri dishes with a concentration of 2×10⁵ cells/dishes. After 24 hrs of the virus AdHNF4α infection, 8×10³ cells from each line were taken and inoculated into a 10 cm Petri dish, and the culture solution was changed every 3 days for 3-4 weeks, until the colonies were visible. The colonies were fixed with 4% PFA, stained with crystal violet, and were counted.

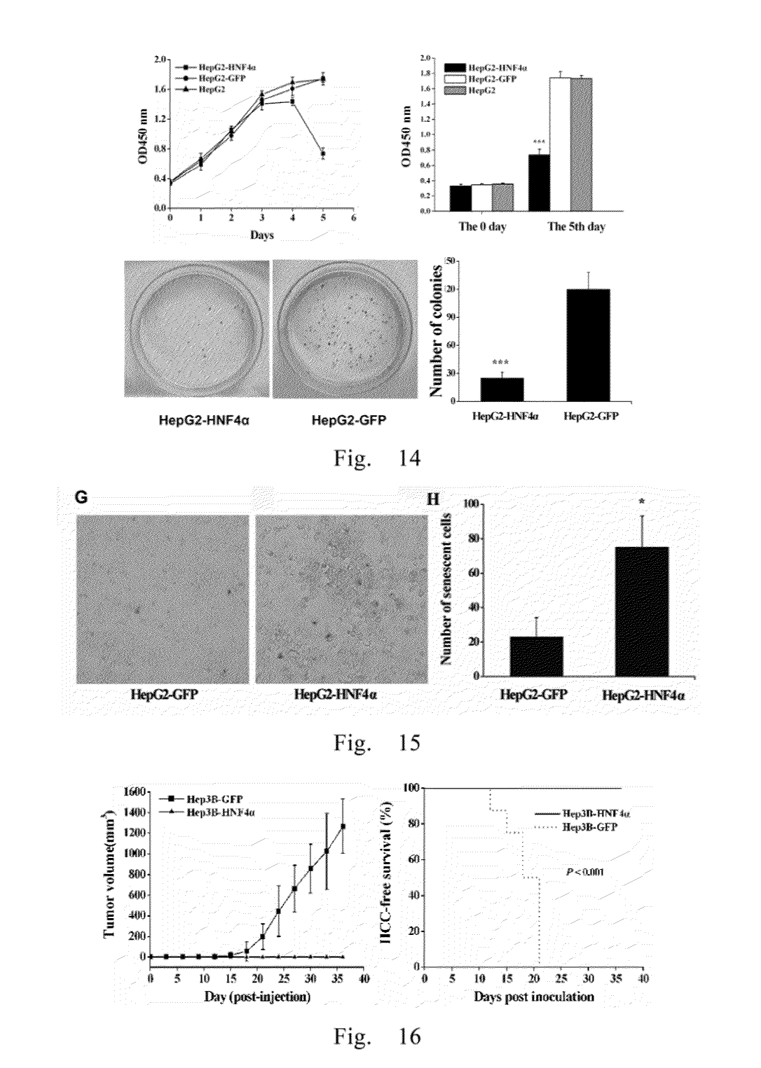

The results showed that, compared to the control group, the colonies formed by the human solid tumor cell lines after AdHNF4α infection were decreased, and the up-regulated expression of HNF4α significantly reduced the capability of solid tumor cell lines for forming colonies (FIGS. 13 and 14).

### Example 8

### Effects of Exogenous HNF4α on the β-Gal Staining Related to Human Hepatoma Cell Aging

The Hep3B and HepG2 were independently inoculated onto the six-well plate with a concentration of 2×10⁵ cells/dishes. The cells were fixed with 4% PFA after the infection for 72 h, 96 h, respectively, and then stained with the age-related β-gal staining solution (newly prepared) at 37° C. for 4-6 hrs. The cells were washed by PBS, and the photographs were taken under an optical microscope. Solution formula: 1 mg/ml 5-bromo-4-chloro-3-indolyl β-D-galactoside (X-Gal), 40 mM citric acid/sodium phosphate (pH 6.0), 5 mM K₄Fe (CN)₆ (potassium ferrocyanide), 5 mM K₃Fe(CN)₆ (potassium ferricyanide), 150 mM NaCl, and 2 mM MgCl2.

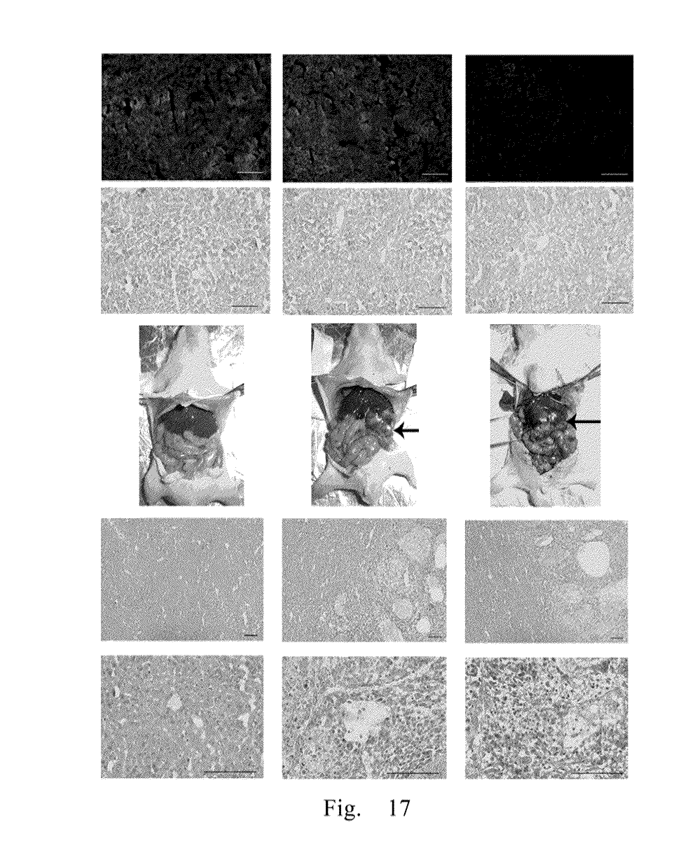

The results showed that, after the HNF4α gene transfer, the positive cells with β-gal staining in HepG2 cells group were significantly increased, suggesting that the up-regulated HNF4α inhibited some liver tumor cells through the induction of aging (FIG. 15).

### Example 9

### Effects of HNF4α Up-Regulation in Human Hepatoma Cell Lines HepG2 and Hep3B on the Formation of In Vivo Tumor

5×10⁶ cells of Hep3B and 1×10⁷ cells of HepG2 which were infected by AdHNF4α for 24 h were taken and inoculated under the armpit of nude mice to observe tumor growth in vivo. The size of neogenesis tumor was measured with a vernier caliper.

The results showed that, for Hep3B control group, the tumor growth was detectable as early as the second week after inoculation, and the tumor growth was detectable in all mice at the third weeks. For HepG2 control group, the tumor growth was detectable at the third week after inoculation, and the tumor growth was detectable in 75% of nude mice at the fifth weeks. During the 5 weeks in the observation, no significant tumor growth was observed in the nude mice which were inoculated with the liver tumor cell infected by AdHNF4α (FIGS. 16 and 17).

### Example 10

### The Differentiation Therapy of Experimental Liver Cancer Model Using HNF4α Gene (1)

5×10⁶ of Hep3B were resuspended in 200 μl serum-free MEM, and injected into nude mice through the spleen, while during 0 d and 2 d after the cell injection, 5×10⁹ pfu of AdHNF4α were injected into the animals by intravenous injection. The nude mice were sacrificed after 8 weeks. The liver was removed, and the frozen sections were used for HE staining and pathological analysis.

The results showed that after the virus was injected through intravenous injection for 3 days, the liver was removed and frozen sections was prepared, 80% of GFP expression was observed under fluorescence microscope in liver cells. After 8 weeks, the significant tumor growth in all the livers of the control group was observed, while there were two nude mice without tumor growth in HNF4α gene therapy group, one only with a small tumor. Further analysis of HE staining showed that, in HNF4α gene therapy group, the normal liver tissue structure with HE staining was observed in two nude mice without tumors, but the malignant cells were found in control group.

### Example 11

### The Differentiation Therapy of Experimental Liver Cancer Model Using HNF4α Gene (2)

After having established the experimental model of liver tumor in nude mice using hepatoma cells inoculation by subcutaneously in the neck, 1×10¹⁰ pfu of AdHNF4α was injected through the jugular vein into the animals.

The results showed that, 3 days after virus injection, more than 80% of GFP expression was observed under fluorescence microscopy in tumor cells. 1 week after treatment, the tumor size was regularly determined, the average size of 8 nude mice at different time in HNF4α gene therapy group were significantly lower than that in the control group. As for the comparison of survival time, HNF4α gene therapy group had a significantly longer survival time than the control group. Immunohistochemistry results showed that tumor cell atypia in treatment group was significant changed (the shape was relatively regular, the nucleus was small, and the increase of nuclear abnormalities and karyokinesis were rare), and no significant changes in expression were found for apoptosis-related proteins such as Bcl-2, Bax and so on. These results suggested that, HNF4α gene/protein effectively induced or promoted the differentiation of solid tumor to normal cells.

### Example 12

### In Vitro Effects of all-Trans Retinoic Acid, Somatostatin, Tumor Necrosis Factor and Arsenic Trioxide on Hepatoma Carcinoma Cell Lines HepG2 and Hep3B

Human hepatoma cell lines HepG2 and Hep3B were inoculated onto the six-well plate with a concentration of 5×10⁵ cells/dish, and the all-trans retinoic acid, somatostatin, tumor necrosis factor, and arsenic trioxide were added respectively. The total RNA was extracted by Trizol kit, and reverse transcriptase reaction was carried out for 2 hrs. 1 μl diluted reverse transcription product was taken and used as template for PCR amplification to detect the mRNA expression of liver cell-related function genes. The proliferation and apoptosis related proteins such as Cyclin, Bax, Bcl-2 and so on was measured by immunohistochemistry.

The results showed that no significant difference was observed in liver cell related function genes for each group, no significant up-regulation was observed in the expression of certain tumor differentiation-related genes (HNF4α, HNF1α, C/EBP). No significant changes in cell morphology was observed. In the tumor necrosis factor group and the arsenic trioxide group, the apoptosis was significantly increased and proliferation was reduced. These results suggested that these control substances had no effect of differentiation induction on the liver cancer cells because there was no change in cell morphology.

Discussion

Hepatocyte nuclear factor 4 (HNF4) is a transcription factor of cell nuclear hormone receptor family, which is an important transcription protein for regulation of hepatocyte differentiation and maintenance of hepatocyte biological functions. It is highly expressed in differential and mature hepatocyte and HNF4α is the important subtype of HNF4. The acession number of wild-type human HNF4α sequences is Gene ID: 419198.

The study on the HNF4α gene knockout mice showed that there was a large number of down-regulated function gene expression in the different developmental stages of liver cells, these genes not only affected the differentiation phenotype of liver cells, but also affected the important gene expression in liver cells involved in fat metabolism, albumin synthesis and drug detoxification and others. HNF4α combines with cis-acting element in form of dimer; when DNA and binding domain of HNF4α form dimer with pregnant X receptor (PXP), it identifies DNA sequences using zinc-finger DNA-binding domain and regulates its own activity by acetylization, phosphorylation and binding with SMADS 3 or 4. It can also interact with activator protein (e.g., SRC-1, GRIP-1 and CBP/p300) to alter the chromosome structures near promotor or enhancer so as to realize regulation of differentiation and functional gene expression at the transcriptional level.

A vast majority of materials or genes in vitro may improve the biological characteristics of some tumor cells or reduce the animal tumor formation of the cancer cells in vivo, but they are almost achieved through the induction of apoptosis, and do not induce specifically in vivo the differentiation of solid tumor. Therefore, although earlier studies showed that the up-regulation expression of HNF4α in liver cancer cell lines can improve some biological characteristics of the tumor cells, no one believes and no one has ever confirmed that HNF4α have the capability of differentiation induction of the malignant solid tumors and reverse the poor differentiation state of tumors. Whether HNF4α has differentiation regulation effects on malignant solid tumors is unclear and the up-regulated expression of HNF4α has not yet been studied as a measure for treatment of differentiation induction.

The innovative study results of this invention have shown that gene expression of HNF4α regulated by using genetic engineering techniques in solid tumor cells can effectively induce the differentiation of tumor cells. HNF4α can regulate the expression of many cell differentiation genes and function genes. For example, the expression of some important function genes (such as apolipoproteins, aldolase B, phenylalanine hydroxylase, TFN and retinol binding protein) is significantly increased by the up-regulation of HNF4α expression in the embryonic stem cells.

More importantly, the up-regulated expression of HNF4α can reverse the dedifferentiation state of hepatoma carcinoma cells. Thus, it suggests that HNF4α may also play an important role in the differentiation and transcription regulation in the different types of tumors. Therefore, the present invention has identified that up-regulated expression of HNF4α by the injection of HNF4α adenovirus vector has the treatment effects of differentiation induction in vivo in animal models of human malignant solid tumor, thereby providing a new treatment measure for tumor differentiation induction.

All the documents cited herein are incorporated into the invention as reference, as if each of them is individually incorporated. Further, it would be appreciated that, in the above teaching of the invention, the skilled in the art could make certain changes or modifications to the invention, and these equivalents would still be within the scope of the invention defined by the appended claims of the present application.

1-10. (canceled)

11. A method for inducing or improving the differentiation of solid tumor in mammal, which comprises a step of administrating HNF4α protein, its coding sequence or expression vector containing said coding sequences to a mammal subject in need of.

12. The method of claim 1 wherein the expression vector comprises viral vector and non-viral vector.

13. The method of claim 1 wherein the solid tumor is selected from the group consisting of liver cancer, gastric cancer, colon cancer, pancreatic cancer, lung cancer, prostate cancer and genital tumor.

14. The method of claim 1 wherein the HNF4α is human HNF4α.

15. The method of claim 1 wherein the administrating comprises intratumoral, intramuscular, intravenous, subcutaneous, intradermal or topical administrating

16. The method of claim 1 wherein the method further comprises a step of administrating a chemotherapeutic agent to said mammal subject.

17. The method of claim 1 wherein the mammal subject is human.

18. A method for inducing differentiation of tumor cells in solid tumor in mammal, which comprises a step of administrating HNF4α protein, its coding sequence or expression vector containing said coding sequences to a mammal subject in need of.

19. A method for preparing a pharmaceutical composition useful for inducing differentiation of malignant solid tumor cells in mammal, wherein the method comprises a step of mixing (a) HNF4α protein, HNF4α coding sequence or expression vector containing said coding sequence with (b) a pharmaceutically acceptable carrier or excipient, thereby forming the pharmaceutical composition

20. A use of Hepatocyte Nuclear Factor 4α or HNF4α gene and/or protein in the preparation of differentiation induction agent or composition for inducing the differentiation of malignant solid tumor cells.


SEQUENCE LISTING (22 sequences)

SEQ ID NO: 1 - DNA, 22 bp, Artificial
  misc_feature: primer
ttgaaaatgtgcaggtgttgac

SEQ ID NO: 2 - DNA, 22 bp, Artificial
  misc_feature: primer
cagagatgggagaggtgatctg

SEQ ID NO: 3 - DNA, 19 bp, Artificial
  misc_feature: primer
gggtactccttgttgttgc

SEQ ID NO: 4 - DNA, 21 bp, Artificial
  misc_feature: primer
aaatcccagaactcagagaac

SEQ ID NO: 5 - DNA, 20 bp, Artificial
  misc_feature: primer
ggctccatgactgtgggatc

SEQ ID NO: 6 - DNA, 20 bp, Artificial
  misc_feature: primer
ttcagctgcacagcccagaa

SEQ ID NO: 7 - DNA, 20 bp, Artificial
  misc_feature: primer
agcctaaggcagcttgactt

SEQ ID NO: 8 - DNA, 20 bp, Artificial
  misc_feature: primer
ctcgatgaacttcgggatga

SEQ ID NO: 9 - DNA, 20 bp, Artificial
  misc_feature: primer
cctgcttgtatgctggagtc

SEQ ID NO: 10 - DNA, 20 bp, Artificial
  misc_feature: primer
gaaaagtcgttgatgttgga

SEQ ID NO: 11 - DNA, 20 bp, Artificial
  misc_feature: primer
ctggcctctgccatcttctg

SEQ ID NO: 12 - DNA, 21 bp, Artificial
  misc_feature: primer
ttagcctccttgctcacatgc

SEQ ID NO: 13 - DNA, 22 bp, Artificial
  misc_feature: primer
gtgtccctctagtctatgaagc

SEQ ID NO: 14 - DNA, 22 bp, Artificial
  misc_feature: primer
attgacttgatcctccagatac

SEQ ID NO: 15 - DNA, 22 bp, Artificial
  misc_feature: primer
gcgggactggtatttgtgtctg

SEQ ID NO: 16 - DNA, 21 bp, Artificial
  misc_feature: primer
ttagtgacgacagccgtggtg

SEQ ID NO: 17 - DNA, 20 bp, Artificial
  misc_feature: primer
agcttggtggtggatgaaac

SEQ ID NO: 18 - DNA, 20 bp, Artificial
  misc_feature: primer
ccctcttcagcaaagcagac

SEQ ID NO: 19 - DNA, 18 bp, Artificial
  misc_feature: primer
catcctgcgtctggacct

SEQ ID NO: 20 - DNA, 20 bp, Artificial
  misc_feature: primer
gtacttgcgctcaggaggag

SEQ ID NO: 21 - DNA, 30 bp, Artificial
  misc_feature: primer
ccgagatctagaatgcgactctccaaaacc

SEQ ID NO: 22 - DNA, 32 bp, Artificial
  misc_feature: primer
cgcgatatcggcttgctagataacttcctgct